(19) United States
(12) Patent Application Publication
(10) Pub. No.: US 2004/0203036 A1
(43) Pub. Date: Oct. 14, 2004

(54) Multimer polynucleotide synthesis

(76) Inventors: Klaus-Peter Stengele, Pleiskirchen (DE); Wolfgang Pfleiderer, Konstanz (DE)

Correspondence Address: GRAYBEAL, JACKSON, HALEY LLP, 155 - 108TH AVENUE NE, SUITE 350, BELLEVUE, WA, 98004-5901, US

(21) Appl. No.: 10/754,447

(22) Filed: Jan. 9, 2004

Related U.S. Application Data
(63) Continuation-in-part of application No. PCT/EP02/07657, filed on Jul. 9, 2002.

(30) Foreign Application Priority Data
Jul. 16, 2001 (DE) 101 33 779.5
Jul. 9, 2001 (DE) 101 32 536.3

(51) Int. Cl. C12Q 1/68; C07H 21/04; C12P 19/34

(52) U.S. Cl. 435/6; 435/91.2; 536/25.3

(57) ABSTRACT

The present invention relates to a process for the preparation of polynucleotides, whereby under suitable usual conditions the free 5′-hydroxy group, whose terminal 3′-hydroxy group contains a usual protecting group, is reacted with a hydroxy group, derivatized in a previous reaction step to a phosphite amidoester, phosphpotriester or phosphonic acid ester, whereby said hydroxy group is a 3′-hydroxy function of a free or solid phase bound polynucleotide, or a solid phase bound hydroxy function.

Further the present invention relates to a kit for performing a process according to the invention, which contains at least one or more selected oligonucleotides, having a free 5′-hydroxy group and a protected 3′-hydroxy group. Further on, the present invention relates to new oligonucleotides and their use as building blocks for the synthesis of polynucleotides in the process according to the invention.

Furthermore the present invention relates to the use of the process according to the invention or the use of the kits for the preparation of poly/oligonucleotides resp. polynucleotide libraries or nucleic acid chips.

The present invention relates to a process for the preparation of polynucleotides, whereby under suitable conditions the free 5′-hydroxy group of selected oligonucleotides, whose terminal 3′-hydroxy group contains a usual suitable protecting group, is reacted with a hydroxy group, derivatized in a previous reaction step to a phosphite amidoester or to a phosphonic acid ester, whereby said hydroxy group is a 3′-hydroxy function of a solid—phase bound polynucleotide, or a solid phase bound hydroxy function.

Further the present invention relates to a kit for performing a process, according to the invention, which contains at least one or more selected oligonucleotide(s) having a free 5′-hydroxy group and a protected 3′-hydroxy group.

Further the present invention relates to the use of the process or kits, according to the invention, for the preparation of oligonucleotides or nucleic acid chips.

Synthetic oligonucleotides are used in all areas of gene technology, for example in gene transfection or in gene analysis. Polynucleotides are prepared by chain extension of a starting compound with many individual nucleoside building blocks. For the synthesis the reacting hydroxy groups are derivatized in such a manner as to form a phosphodiester group, a phosphotriester group or an H-phosphonate group. Other functional groups of the starting compounds, interfering with this reaction, will have usual suitable protecting groups.

For instance DE 199 15 867 A1 and DE 199 38 092 A1 describe photolabile protecting groups for hydroxy groups, which can be introduced into a nucleoside or nucleotide with high yields and release the protected hydroxy group when irradiated.

Nowadays, polynucleotides are prepared primarily by using solid phase techniques in order to optimize the efficiency of the process. The starting compounds are bound either directly or via linkers to functionalized solid surfaces of polymer beads or to glass-, metal- or plastic surfaces and reacted with reagents required for extending polynucleotide chains.

Excess reagents as well as soluble reaction byproducts and solvents can easily be removed from the solid phase bound polynucleotide compounds.

A disadvantage of the known processs is that the plurality of the individual reaction steps lead to low overall yield, even when their individual yield is high.

Therefore, depending on the desired length of the nucleotide chain and the number of individual reaction steps, an excess of starting compounds and reagents has to be used, which require after completed reaction a complex process for (possibly ineffective) re-use. Moreover numerous and often similar undesired byproducts have to be separated from the end product.

Thus a person skilled in the art has to use significant amounts of starting compounds and has to perform complex purification processes.

The problem of the present invention is to provide a process, which does not have the disadvantages of the current state of the art. Such a process in particular should be suitable for automated solid phase synthesis of polynucleotides.

The problem is solved by a process for the preparation of polynucleotides comprising the following steps:

a) The reaction of the free 5′-hydroxy group of a selected oligonucleotide, whose terminal 3′-hydroxy group contains a usual suitable protecting group, with a hydroxy group derivatized in a preceeding reaction step to a phosphite amidoester or to a phosphoric acid ester or to an H-phosphonate, whereby said hydroxy group is a 3′-hydroxy group of a free or solid phase bound polynucleotide or a solid phase bound hydroxy group under suitable conditions and if necessary with the purification of the reaction product,

b) where applicable the oxidation of the reaction product to a phosphodi- or phosphotriester according to step a), if a hydroxy group was used, which has been derivatized to a phosphite amidoester or an H-phosphonate and purification of the reaction product if necessary.

c) Removal of the 3′-hydroxy protecting group of the reaction product according steps a) or b) under suitable conditions and purification of the reaction product if necessary.

d) Where necessary, derivatization of the released 3′-hydroxy group to a phosphite amidoester or in an H-phosphonate according to step c) by using the appropriate usual reagents.

e) if necessary rerun steps a) to c) by using the reaction product activated according step d),

whereby the oligonucleotides with the free 5′-hydroxy function according to step a) are selected in such a way that the desired polynucleotide is obtained.

In the following by “suitable conditions” and “usual suitable conditions” those reaction conditions are understood, which are familiar to the person skilled in the art, as e.g. solvents, temperature, catalyst, energy input (thermally or by radiation), which result in a high coupling resp. cleavage yield.

A preferred embodiment of the present invention comprises steps a) to e) of the above described process, where the free hydroxy group or the 3′-hydroxy group of the polynucleotide is present as solid phase phosphite amidoester (or phosphoric acid ester) and is reacted with the free 5′-hydroxy group of a selected oligonucleotide.

By using selected already functionalized oligonucleotides and starting compounds, which contain free and/or derivatized reactive hydroxy groups, intermediate steps are avoided, as they are necessary for polynucleotide chain extensions with individual nucleotides. For instance with selected dinucleotides only ½ of the necessary couplings are required, with trinucleotides only ⅓ of the necessary couplings are required and so forth.

The selected oligonucleotides, as understood according to the invention, are polynucleotides with 2 to 10 nucleosides, which are preferably connected to each other via 3′-5′-phosphoric acid ester bonds. Polynucleotides also comprise oligonucleotides and polynucleotides with more than 10 nucleotide building blocks.

Selected oligonucleotides, used according to the invention, are pentanucleotides, preferably tetranucleotides, especially preferred trinucleotides and exceptionally preferred dinucleotides.

The process is also suitable for the preparation of extra long polynucleotides according to the so-called “block condensation process”, resp. for preparation of large quantities of polynucleotides according to the block condensation process, since unreacted educts can easily be retrieved and reused in later syntheses or synthesis steps.

The selected oligonucleotides can for instance be composed of nucleoside building blocks according to formula (X), which are connected via 3′-5′-phosphoric acid ester bonds:

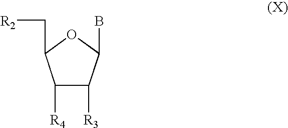

where B can be an H, adeninyl, cytosinyl, guaninyl, thyminyl, uracilyl, 2,6-diaminopurin-9-yl, hypoxanthin-9-yl, 5-methylcytosin-1-yl, 5-amino4-carboxylimidazol-1-yl or 5-amino-4-carbamoylimidazol-1-yl, whereby existing primary amino functions can be protected by a permanent protecting group, resp. thyminyl or uracilyl at the O₄-position can contain a permanent protecting group,

and where R₂ can be a phosphoric acid ester rest, a free hydroxy group, a phosphite amidoester, a phosphonic acid rest or another suitable hydroxy protecting group,

R₃ can be an H, OH, halogen, acylamino-, alkoxy- or alkoxyalkyl rest with 1 to 4 C-atoms,

R₄ can be a phosphoric acid rest, a free hydroxy group, a phosphite amidoester rest, a phosphoric acid ester rest, an H-phosphonate rest or a hydroxy protecting group.

The synthesis of selected oligonucleotides can be performed for instance according to FIG. 1a. Even though this leads to a dinucleotide, it is clear that also tri-, tetra-, penta- and even higher nucleotides can be prepared this way.

In step I the free 3′-hydroxy function of a nucleoside compound A, which has a nucleobase B₂ and whose 5′-hydroxy function is protected by a dimethoxytrityl protecting group, can be derivatized with NPPOC-chloride and thus be protected.

In step II the dimethoxytrityl protecting group (DMT) of the compound B, which thus has been prepared, is cleaved under acid conditions and the 5′-hydroxy function is released.

In step III the free 5′-hydroxy function of compound C is reacted with the 3′-hydroxy function of the nucleoside D, which had been previously derivatized to a phosphite amidoester (R₁ can be for instance C₁ to C₄). In compound C in FIG. 1a L stands for NPPOC, FMOC and NPC. Nucleoside D has a dimethoxytrityl-protecting group (DMT) at the 5′-end and a base Be. After oxidation of the phosphite ester bond into a phosphate ester bond and after cleavage of the 5′-terminal DMT-group, the resulting dinucleotide E, whereby L stands for NPPOC, FMOC and NPC, can now directly be used as selected oligonucleotide in a process according to the invention. Other dinucleotides or oligonucleotides with other bases are available by selecting the corresponding starting compounds C and D.

A dinucleotide E can also be transformed into other long chain oligonucleotides by repeated transformation with nucleosides of e.g. compound D. Further a dinucleotide E can also be transformed with other oligonucleotides into new oligonucleotides or polynucleotides, whose terminal 3′ and 5′-hydroxy group are derivatized equally or functionally equal as compound D.

Another synthesis for polynucleotides according to the invention is shown in FIG. 1b, where instead of a NPPOC-protecting group in 3′-position a p-nitrophenyloxycarbonyl-protecting group is used, whereby Y═O,S. After cleavage of the DMT-protecting group in 5′-position under acidic conditions and reacting with the phosphite amidoester-derivatized mononucleoside (D), an oxidation of the phosphite ester bond into the phosphate ester is performed.

With the reaction scheme according to FIG. 1b, as opposed to FIG. 1a, the DMT-protecting group in 5′-position is cleave only after completed reaction with (J), as it is described in the following in more detail.

For the preparation of polynucleotides, compound (J) in contrast to compound (E), is reacted with the compound XI below only after completed reaction, while being activated with DMAP,

whereby the methyl group can also be an H, and wherein X=O,S or HNR₃, and where R₃=H, alkyl, aryl or aralkyl , in the process according to the invention and after cleavage of the 5′-DMT-protecting group. In FIG. 1b, compound IX is represented but not limited to NPPOH.

A subsequent cleavage of the DMT group under acidic conditions leads to compound (N).

However, all protecting groups commonly used by a person skilled in the art are suitable as intermediary protecting groups of the 3′-hydroxy function. These are protecting groups, which are orthogonal to DMT and cleavable from the permanent base protecting groups, especially photolabile protecting groups.

Preferred photolabile protecting groups of the 3′-hydroxy function are NPPOC, MeNPOC, NPES, NPPS, PyMOC, NVOC, and NBOC. Reagents like e.g. the corresponding chlorides or alcohol are used analogously for the introduction of these protecting groups.

For example as described in FIG. 2, derivatized hydroxy functions of the type phosphite amidoester F (or D) or H-phosphonate resp. H-phosphonate salts (K) or phosphoric acid ester G, whereby A is a halogene selected from F, Cl, Br and R and where R can be an H, alkyl, cycloalkyl, aryl, aralkyl, haloalkyl, cyanoalkyl rest, and if R=H , the compound is preferably a soluble phosphorus diester salt, resp. in the form of a quartemary ammonium salt and n=0 or an integer from 1 to 4, are used for the preparation of polynucleotides according to the invention.

In FIG. 2 X represents an example of a nucleoside or nucleotide rest, a oligo- or polynucleotide rest, a solid phase linker, a hydroxylic derivatized solid phase surface or other possible compounds, which contain a hydroxyl group. Such starting compounds can be freely soluble or bound to solid phases.

The starting compounds can be either soluble or solid phase bound nucleosides or polynucleotides, whose terminal 3′-hydroxy function is a phosphite amidoester, a phosphonic acid ester or an H-phosphonate. Also hydroxy functions of the solid phase itself or its linkers can serve as starting compound, in the form of phosphite amidoester derivatives or phosphoric acid derivatives (G) or H-phosphonates (K).

Besides the selected oligonucleotides also selected mononucleosides can be used in a supplementary way or predominantly, in order to obtain the desired nucleotide chain, if one or another of the necessary oligonucleotides is not available.

In this case the starting compounds derivatized as phosphite amidoester (F) or phosphoric acid ester (G) resp. (K) can then react with a selected oligonucleotide, for instance compound (E) (FIG. 2) by forming the desired polynucleotide in steps IV, Va and Vb. In compound E in FIG. 2 L stands for NPPOC, FMOC and NPC. The phosphite amidoester must be activated with 1-H-tetrazol (TET) or 4,5-dicyanoimidazol (DCI) in acetonitrile before the reaction. The H-phosphonate salt (K) is activated before the reaction Vb with pivaloylchloride or adamantoylchloride in triethylamine/acetonitrile. The coupling product is either the end product or an intermediary product, which still has to be extended.

If the desired end product has already been obtained, just the protecting groups at the terminal 3′-end, here a NPPOC-protecting group, as well as all so-called permanent protecting groups have to be cleaved, if necessary after oxidation of the trivalent phosphorus (FIG. 2, step VI).

If the elongated intermediary product (E) shall be extended even further, the deprotected 3′-hydroxy group is derivatized again in step VII to form the phosphite amidoester H or the corresponding phosphonic acid ester and is reacted with another oligonucleotide E. For this the NPPOC-protecting group must first be transformed into a hydroxy function (Step VI). This reaction sequence is repeated, by varying the selected oligomers and if necessary some individual nucleosides (derivatized correspondingly), as often as necessary until the desired polynucleotide is obtained (step VIII).

Furthermore the problem of the present invention is solved by providing new nucleotide derivatives (L), (E), (M) and (J). These are preferably used as building blocks in the described process according to the invention.

The nucleotide derivative (L) has the following general formula,

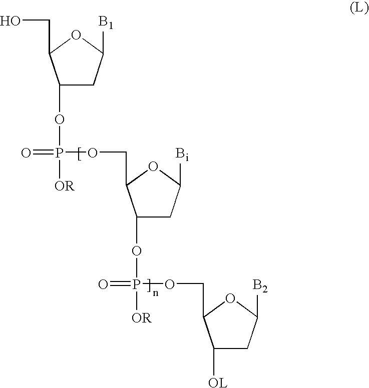

where B₁, B₂, Bᵢ can be H, adeninyl, cytosinyl, guaninyl, thyminyl, uracilyl, 2,6-diaminopurin-9-yl, hypoxanthin-9-yl, 5-methylcytosin-1-yl, 5-amino-4-carboxylimidazol-1-yl or 5-amino-4-carbamoylimidazol-1-yl, independently from each other, and in the case of B₁, B2, Bᵢ, where a primary amino function is present, can have a permanent protecting group resp. with thyminyl or uracilyl at the O₄-position can have a permanent protecting group if necessary,

and where R can be an H, alkyl, cycloallryl, aryl, aralkyl, haloalkyl, cyanoallryl rest, and if R=H, the compound is preferably a soluble phosphorus diester salt, resp. in the form of a quartemnary ammonium salt and n=0 or an integer from 1 to 4, and where L stands for NPPOC, FMOC and NPC.

The nucleotide derivative (E) has the following general formula:

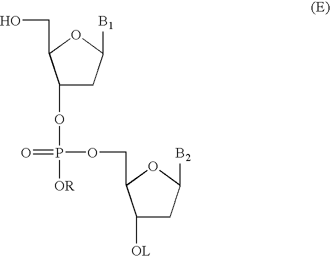

where B, and B₂ can be H, adeninyl, cytosinyl, guaninyl, thyminyl, uracilyl, 2,6-diaminopurin-9-yl, hypoxanthin-9-yl, 5-methylcytosin-1-yl, 5-amino4-carboxylimidazol-1-yl or 5-amino4-carbamoylimidazol-1-yl independently from each other, and in the case of B₁, B₂, where primary amino functions are present, can have a permanent protecting group resp. with thyminyl or uracilyl at the O₄-position can have a permanent protecting group if necessary,

and where R can be an H, alkyl, cycloalkyl, aryl, aralkyl, haloalkyl, cyanoalkyl rest. If R=H, the compound is preferably a soluble phosphorus diester salt, resp. in the form of a quarternary ammonium salt, and where L stands for NPPOC, FMOC and NPC.

The nucleotide derivative (M) according to the invention has the following formula:

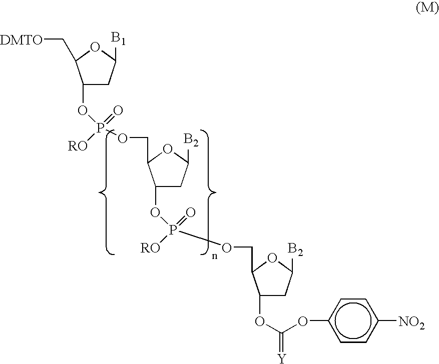

where B₁, B₂, Bᵢ can be H, adeninyl, cytosinyl, guaninyl, thyminyl, uracilyl, 2,6-diaminopurin-9-yl, hypoxanthin-9-yl, 5-methylcytosin-1-yl, 5-amino-4-carboxylimidazol-1-yl or 5-amino-4-carbamoylimidazol-1-yl independently from each other, and in the case of B₁, B₂, Bᵢ, where primary amino functions are present, can have a permanent protecting group resp. with thyminyl or uracilyl at the O₄-position can have a permanent protecting group if necessary. If R=H, the present compound is preferably a soluble phosphorus diester salt, resp. in the form of a quartemary ammonium salt.

and where R can be an H, alkyl, cycloalkyl, aryl, aralkyl, cyanoalkyl, haloalkyl rest. If R=H, the compound is preferably a soluble phosphorus diester salt, resp. in the form of a quartemary ammonium salt.

and Y═O or S and n=0 or an integer from 1 to 4.

The nucleotide derivative (J) has the following formula:

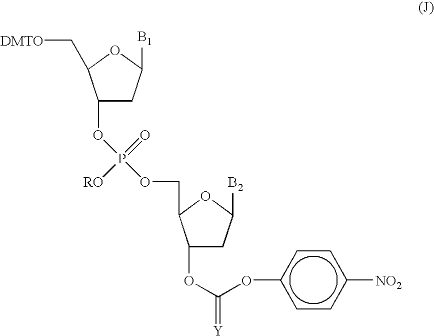

where B₁ and B₂ can be H, adeninyl, cytosinyl, guaninyl, thyminyl, uracilyl, 2,6-diaminopurin-9-yl, hypoxanthin-9-yl, 5-methylcytosin-1-yl, 5-amino-4-carboxylimidazol-1-yl or 5-amino-4-carbamoylimidazol-1-yl independently from each other, and in the case of B₁, B₂, Bᵢ, where primary amino functions are present, can have a permanent protecting group resp. with thyminyl or uracilyl at the O₄-position can have a permanent protecting group if necessary,

R can be an H, alkyl, cycloalkyl, aryl, aralkyl, haloalkyl, or cyanoalkyl rest. If R=H, the present compound is preferably a soluble phosphorus diester salt, resp. in the form of a quarternary ammonium salt.

and Y═O or S.

The use of a p-nitrophenyl-O—C(Y)-protecting group advantageously facilitates to avoid the highly toxic and dangerous phosgene during introduction of protecting groups.

The use of the dinucleotides (E) and (J) according to the invention resp. of the oligonucleotides (L) and (M) according to the invention, in the process according to the invention permits a fast and specific synthesis of long polynucleotides. At the same time higher selectivity and higher yield are obtained, since intermediary steps, as they weren necessary, with the previous processes according to the state of the art with the use of mononucleotides do not apply.

The building blocks needed for chain extension are more stable and have a longer shelf life than building blocks of the state of the art and can be recovered after the reaction, as the activation takes place at the solid support in contrast to the state of the art, where the incoming building block, which is usually present in a 2 to 9 fold molar excess over the growing oligonucleotide, is activated to a highly reactive yet unstable intermediate, the excess being discarded after the coupling step. As the compounds of the invention are not activated, any excess material can be reused in subsequent syntheses or synthesis steps even without further purification. The so-called capping step does not apply, since the 3′-hydroxy functions, directly transformed into phosporus III, in subsequent synthesis steps, after activation but without completed coupling, cannot be extended. The reaction therefore substitutes the capping step completely. This is the case above all, because the reactivity of the reagents used for phosphitization is higher than that of the so-called capping reagents.

The compounds, which have a hydroxy function derivatized as phosphite amidoester or phosphonic acid ester, are preferably bound to a solid phase.

Preferred solid phases are carrier materials made from silica gel, glass, metal, preferably magnetic metal, plastic, cellulose, dextrane cross-linked with epichlorohydrine, agarose, styrene-divinylbenzene resins, preferably 4-(Hydroxymethyl)-phenoxymethyl-copolystyrene-divinylbenzene resins or chloromethylated Co-polystyrene-divinylbenzene resin, especially preferred styrene-divinylbenzene resins with 1% divinylbenzene content.

Plastic carrier materials comprise plastic films resp. membranes made of polypropylene, Nylon, cellulose, cellulose derivatives, for instance cellulose acetate, cellulose-mixed ester, polyether sulfones, polyamides, polyvinylchloride, polyvinyliden fluoride, polyester, Teflon or polyethylene.

The carrier surface can contain free or protected functional groups, e.g. amino-, hydroxyl-, carboxyl-, carbonyl-, thiol-, amide- or phosphate groups. Such groups can also be connected with the polynucleotide via a linker molecule.

Planar carrier surfaces are used as nucleic acid chips. Nucleic acid chips, according to the invention, are biomolecules built on a solid carrier, like DNA or RNA, and nucleic acid analogs, like PNA, LNA or chimeras of those with DNA, RNA or nucleic acid analogs.

The process according to the invention is preferably used for the manufacture of nucleic acid chips, where the synthesized polynucleotide is attached to the solid phase via the 5′-end and the 3′-OH group is freely accessible. Such nucleic acid chips are suitable both for hybridization experiments and for certain enzyme reactions (e.g. DNA-ligase, DNA-polymerase), that require a free 3′-OH. By using the process according to the invention nucleic acid chips are available, which are to be prepared faster, in higher yields and also containing longer polynucleotides than using the traditional nucleoside for nucleotide synthesis.

The processes according to the invention are not only suitable for DNA- and RNA-nucleotide synthesis. The synthesis of polynucleotides made of nucleic acid analogs, like PNA, LNA or chimeras of those with DNA, RNA or nucleic acid analogs is also possible.

The processes according to the invention are particularly suitable for implementation in an automated process. Such an automated process is preferably carried out as parallel synthesis for the preparation of a nucleotide library, where the selected oligonucleotides and if necessary some mononucleotides are selected specifically or at random.

It is time saving and useful for the person skilled in the art to generate an oligonucleotide pool of different nucleic acid sequences for use in the process according to the invention. These different nucleic acid sequences should be preferably already derivatized correspondingly, so that they can be used without further processing.

It is particularly advantageous that the unused portion of oligonucleotides can be reused in a subsequent synthesis step without further reconditioning. Furthermore the oligonucleotide building blocks are more stable as compared to the state of the art and therefore have a longer shelf life.

The process according to the invention is also preferably used for large-scale manufacturing of therapeutic nucleic acid analogues in a cost-effective manner. Also, the reduction of the number of chemical unit-operations, like synthesis, washing and drying steps significantly lower the overall-cost of large scale oligonucleotide synthesis. Even more, according to the process, oxidation and cleavage reactions can be performed in one step (VII and VIII).

Another specification of the present invention comprises a kit, which contains part of or all reagents and/or auxiliary supplies and/or solvents and/or work instructions for the implementation of a process according to the invention in one unit. The kit contains at least one or several selected oligonucleotides, which preferably contain a free 5′-hydroxy function and a protected 3′-hydroxy function.

Another embodiment of the present invention comprises the use of processes according to the invention and/or the above mentioned kit for the preparation of oligonucleotides or nucleic acid chips, preferably for the automated preparation of oligonucleotides or nucleic acid chips or nucleic acid analogues in a large scale.

### Abbreviations

DCI 4,5-dicyanoimidazole

DMT dimethoxytrityl—

TsOH toluenesulfonic acid

NPPOC 2-(2-nitrophenyl)propyloxycarbonyl

PYMOC pyrenylmethyloxycarbonyl

MeNPOC 2-(3,4-methylendioxy-2-nitrophenyl)propyloxycarbonyl

NPE 4-nitrophenylethyl

NPC 4-nitrophenyloxycarbonyl

NPS 2-nitrophenylethylsulfonyl

NPPS 2-(2-nitrophenyl)propylsulfonyl

NVOC 2-nitroveratryloxycarbonyl

NBOC 2-nitrobenzyloxycarbonyl

NPPOH 2-(2-nitrophenyl)propanol

DMAP 4-(dimethylamino)-pyridine

FMOC 9-fluorenylmethoxycarbonyl

Bz benzoyl

### FIGURES

FIG. 1a and 1b show illustrative, non limiting examples of synthesis schemes for the preparation of selected oligonucleotides,

FIG. 2 shows an example of an illustrative non limiting synthesis scheme for the preparation of polynucleotides according to the invention using a process according to the invention,

FIG. 3 shows a further non-limiting synthesis scheme for oligonucleotide dimers according to the invention.

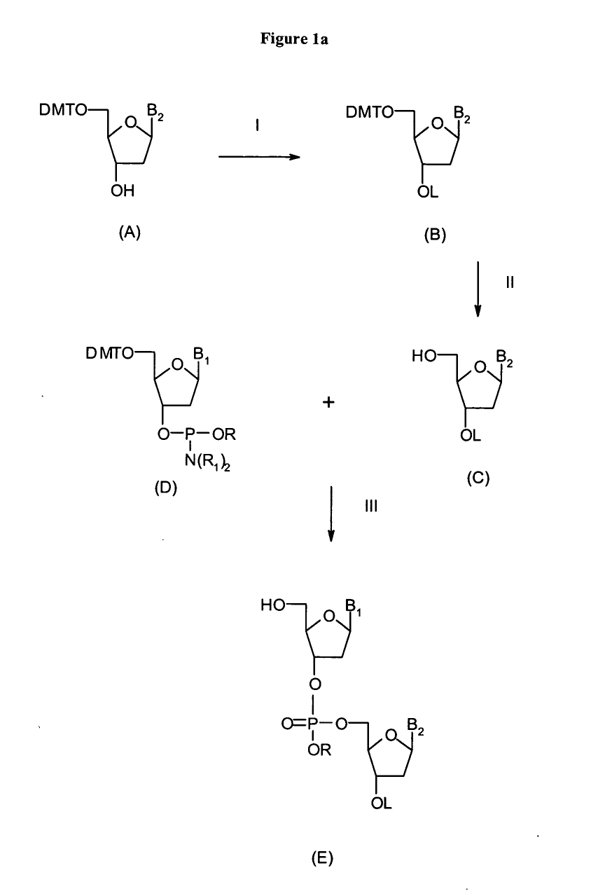

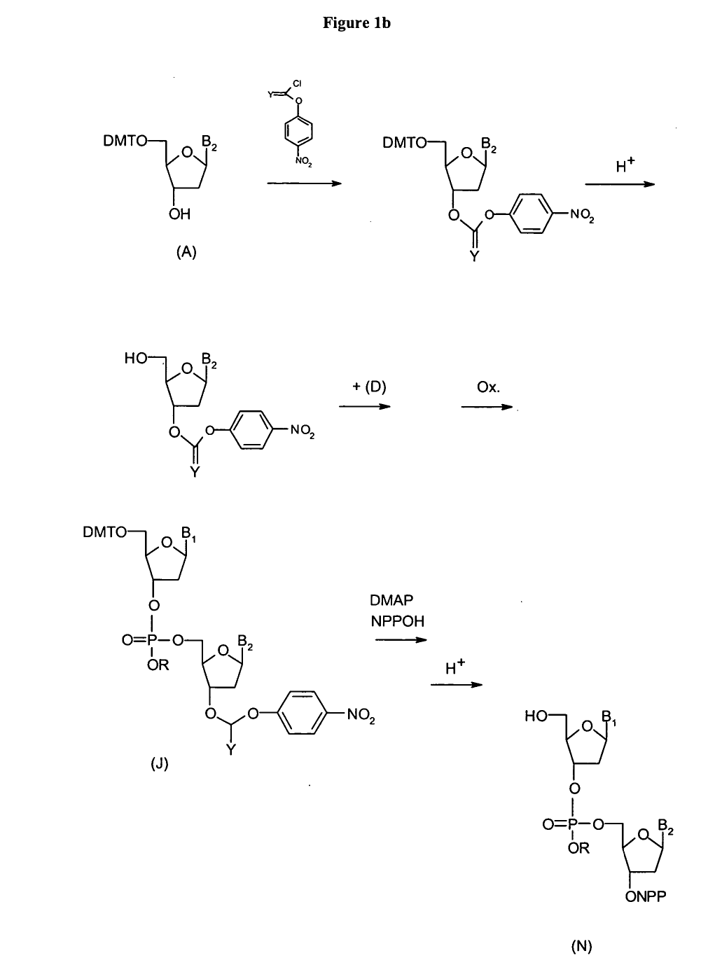

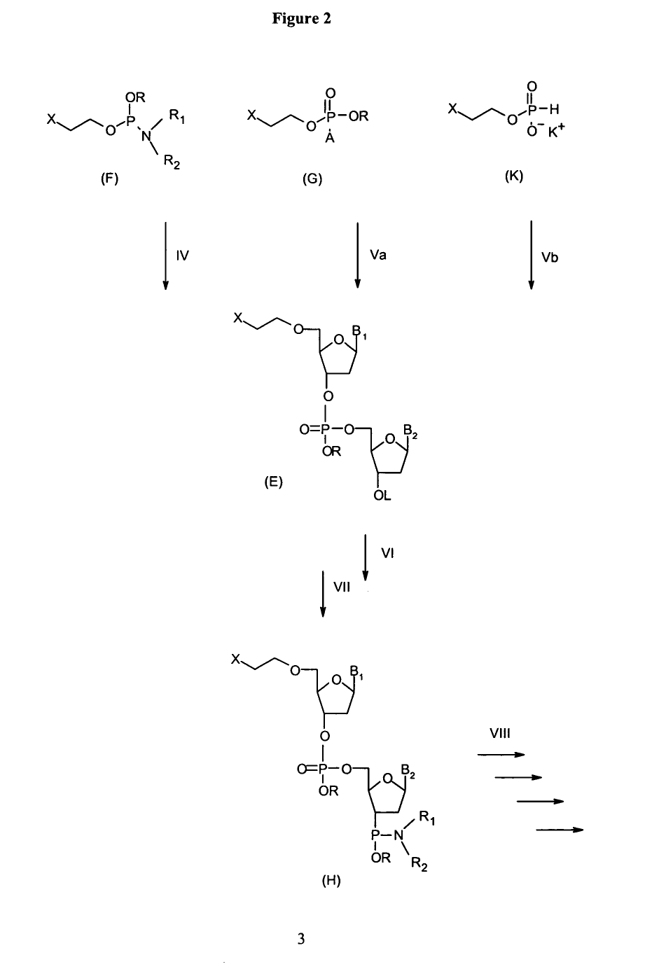

FIGS. 1 and 2 are explained in detail in the foregoing-description.

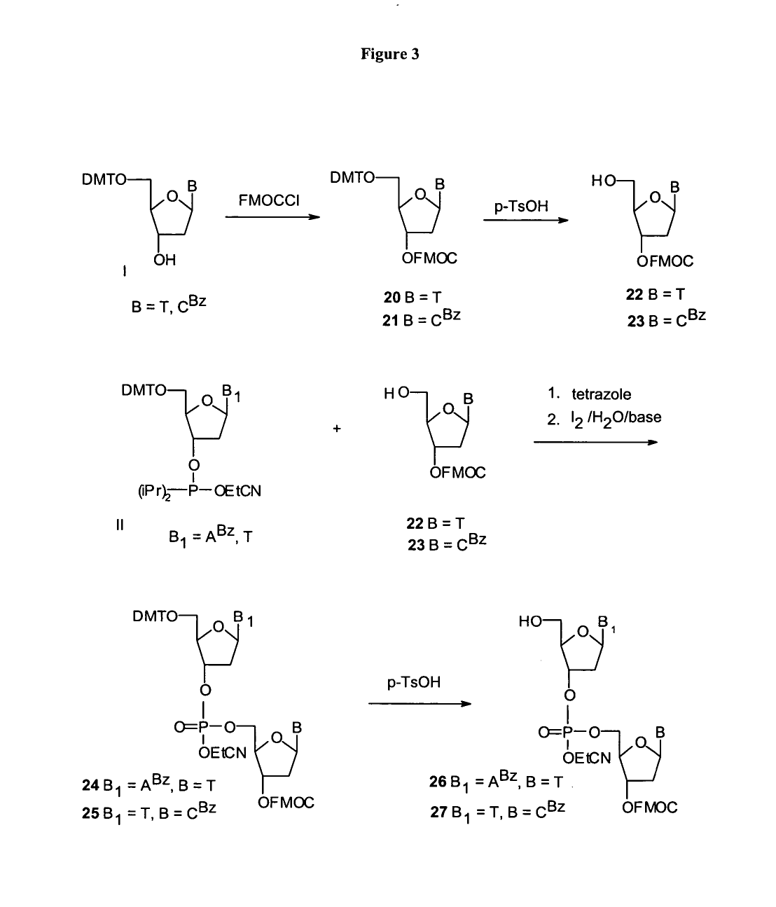

FIG. 3 shows another exemplary embodiment of the invention for the synthesis of oligonucleotides according to the invention, namely a number of 3′FMOC protected oligodinucleotides synthesized via the process according to the invention.

In a first reaction step, the free 3′-hydroxy function of a nucleoside compound I, which has a nucleobase B, which is thyminyl (T) or benzoyl protected cytosinyl (C^{Bz}) and whose 5′-hydroxy function is protected by a dimethoxytrityl protecting group (DMT), is derivatized with FMOC-chloride and be protected. This reaction step leads to compounds 20 and 21. In the next reaction step, the dimethoxytrityl protecting group (DMT) of the compounds 20 and 21 is cleaved under acid conditions and the 5′-hydroxy function is released, yielding compounds 22 and 23. It is understood that a similar reaction falling under the scope of the invention can be carried out by using compound (J) and the corresponding alcohol like FMOH and the like. The reaction conditions are described in detail in the following examples, but are not limited to those set forth and may be applied and varied according to the needs of a person skilled in the art such applications and variations being also within the scope of the invention. This concerns especially reaction time, solvent, reactive agents, temperature pressure etc.

The next reaction step of the reaction scheme involves the tetrazole-assisted coupling reaction of the free 5′-hydroxy function of compounds 22 or 23 with the 3′-hydroxy function of the nucleoside II, which had been previously derivatized to a phosphite amidoester. It is understood that any other coupling assisting agent known by a person skilled in the art can also be used. Nucleoside II has a dimethoxytrityl-protecting group (DMT) at the 5′-end and a base B₁. which is for example thyminyl (T) or benzoyl protected adeninyl (A^{Bz}). Coupling and oxidation with usual reagents known to a person skilled in the art, for example iodine, of the phosphite ester bond into a phosphate ester bond are performed as subsequent steps in a one-pot reaction leading to compounds 24 and 25. After cleavage of the 5′-terminal DMT-group under acidic conditions, preferably with TsOH or other suitable acids, known by a person skilled in the art, the resulting dinucleotides 26 and 27 can now directly be used as selected oligonucleotide in a process according to the invention. Other dinucleotides or oligonucleotides with other bases are available by selecting the corresponding starting compounds. The reaction conditions for this specific reaction involving FMOC protected oligonucleotides are described in detail in the following examples 15 to 26, but are not limited to those set forth and may be applied and varied according to the needs of a person skilled in the art such applications and variations being also within the scope of the invention. This concerns especially reaction time, solvent, reactive agents, temperature pressure etc.

In the following the present invention is further explained by a number of examples while making reference to the corresponding figures. These examples are meant solely for the explanation and illustration of the invention and do not restrict or limit the general idea underlying the invention.

### EXAMPLES

### Example 1

### Preparation of 3-0-[2-(2-Nitrophenyl)propyloxycarbonyl]thymidine

4.05 g 2-(2-Nitrophenyl)propyloxycarbonylchloride (NPPOC—Cl) (16.6 mmol, 1.3 equivalents) in 5 ml dichloromethane were added to a solution of 7 g 5′-0-Dimethoxytrityl-thymidine (DMTr-Thd) (12. 8 mmol) in 70 ml pyridine under argon protection atmosphere, while being cooled in an ice-water bath. The ice bath was removed and the reaction mixture was stirred at room temperature for 4 hours. Subsequently 2 ml methanol was added to the reaction solution. After another 15 min the reaction mixture was diluted with 250 ml dichloromethane, and washed 2× with 100 ml water. The organic phase was dried over sodium sulfate and evaporated until an oil was obtained. The rest was evaporated with some added toluene until an oil was obtained.

The resulting oil was dissolved in a mixture of dichloromethane and toluene (20+10 ml) and adding it dropwise to 500 ml hexane precipitated the raw product. The precipitate was filtered, dried and reacted for 15 min with a 1% solution of toluene sulfonic acid in dichloromethane/methanol. After the reaction the mixture was made up to a final volume of 300 ml with dichloromethane, washed with 100 ml of a saturated aqueous solution of sodium hydrogen carbonate and 100 ml water, dried over sodium sulfate and evaporated to a final volume of 20 ml. The residual solution thus purified is added dropwise into 500 ml hexane, the resulting precipitate is filtered and washed with hexane. The resulting amorphous solid is finally purified by column chromatography (silica gel, 160 g, column 6×15 cm, washed with 1 liter dichloromethane and then a gradient solution of dichloromethane with methanol 99:1 to 50:1 is applied. 5 Liter of this gradient eluant is collected as main fraction). The product fractions are collected and evaporated. The final product is obtained as 4.5 g foam. The overall yield is 78 mol %.

### Example 2

Preparation of Thymidylyl-{3′-[O⁸-(2-cyanoethyl)]-5′}-3′-O-[2-(2-nitrophenyl) propyloxycarbonyl]) thymidine

A mixture of 1 g 5′-O-Dimethoxytritylthymidine-3′-(2-cyanoethyl-N-diisopropylphosphite amide (1.06 mmol, 1 equivalent), 396 mg 3′-O-[2-(2-nitrophenyl)propyloxycarbonyl]thymidine (0.88 mmol, 0.83 equivalent) and 350 mg tetrazole (5 mmol,) was stirred in 10 ml acetonitrile under argon protection atmosphere and light exclusion for 5 hours at room temperature. The reaction solution was stored over night at −19° C. The next day a 05 molar iodine solution in pyridine/dichloromethane/water (ratio 3:1:1) was added dropwise to the reaction solution until the color remained stable and then the solution was stirred for another 30 min. The reaction solution was mixed with 150 ml dichloromethane, sodium thiosulfate solution was added until decolorization occurred. The solution was dried over sodium sulfate and the solvent was evaporated. The rest was dissolved in toluene and the solvent evaporated. The rest was dissolved in 100 ml of a 1% toluene sulfonic acid solution in dichloromethane and methanol (ratio 9:1). After 15 min the solution was treated with 60 ml 1% sodium hydrogen carbonate solution, washed with 60 ml water, dried with sodium sulfate and evaporated with a rotary evaporator resulting a solid foam. Thinlayer chromatography (hexane /ethylacetate, 1:1) confirmed that no more 3′-NPPOC-Thd was left in the product.

The final purification of the product (1.2 g) was done by column chromatography (80 g silica gel, column 4×17 cm, washed with 0.5 liter dichloromethane, main fraction 3 liter using a gradient of dichloromethane/methanol 49:1 to 9:1).

The main fraction was collected, evaporated and resulted in a solid amorphous foam. The yield was 65%.

### Example 3

### Preparation of2-Bromoethoxy-dichlorophosphane

12.5 g Bromoethanol (0.1 mol, 7 ml) in 10 ml acetonitrile was added to a solution of 16.5 g phosphorus trichloride (0.12 mol, 10.5 ml) in 20 ml acetonitrile at −20° C. and was stirred at this temperature for 45 min. This solution was brought to −5° C. while stirring within one hour and then was stirred for another hour. Subsequently the solvent was evaporated at 30 to 35° C. The rest was dissolved in 15 ml acetonitrile and evaporated again. The final end product obtained (from acetonitrile) was 19.2 g, which corresponds to yield of 85%.

The oily end product was used directly in the synthesis of Example 4 without any further purification.

### Example 4

### Preparation of 2-Bromoethoxy-chloro-diethylaminophosphane

12,4 g Diethyltrimethylsilylamine (85.3 mmol, 16.1 ml) in 10 ml dichloromethane were added within one hour at about −20° C. under argon protection atmosphere to 19.1 g 2-bromoethoxy-dichlorophosphane (84,5 mmol), prepared in Example 4, in 20 ml dichloroethane. The reaction mixture was stirred over night at room temperature. Dichloromethane and the trimethylsilyl chloride formed during the reaction was evaporated at 33° C. under vacuum (2-5 mm Hg). 24 g of the resulting oily yellow reaction product (yield 99 mol %) was used for the phosphitization in Example 5, without further purification.

### Example 5

### Preparation of 5′-O-DMT-Thymidine-3′-O-(2-bromoethyldiethylamino phosphite)

0,51 g Amidite from Example 4 (1.94 mmol, 1.4 equivalents) were added under argon protection atmosphere to a solution of 1 g (1.389 mmol) 5′-O-DMT-Thymidine and 0,63 g (4.86 mmol, 0.83 ml, 3.5 equivalents) diisopropylethylamine in 7 ml dichloroethane. The resulting reaction solution was stirred at room temperature for 4 hours. Subsequently another 0,39 g of the amidite from Example (total of 2.4 equivalents, 3.42 mmol) were added to the reaction solution and stirred for one hour. Then 0.2 ml methanol were added, left for 5 min, diluted with 100 ml dichloromethane, washed with 60 ml of sodium hydrogen carbonate solution and water and dried over sodium sulfate. After the solvent has been removed by evaporation, the resulting 2g of oil were finally purified by column

| | B | R | R¹ | | B¹ | R | | B | B¹ | R | R² |
|---|---|---|---|---|---|---|---|---|---|---|---|
| 1 | T | iPr | EtCN | 7 | T | NPPOC | 11 | T | T | EtCN | NPPOC |
| 2 | T | Et | NPE | 8 | T | NPC | 12 | T | T | EtCN | NPC |
| 3 | dA^{tac} | iPr | EtCN | 9 | dC^{Ac} | NPPOC | 13 | T | dC^{Ac} | EtCN | NPPOC |
| 4 | dA^{Bz} | iPr | EtCN | 10 | dG^{dmf} | NPPOC | 14 | T | dG^{dmf} | EtCN | NPPOC |
| 5 | dC^{Ac} | iPr | EtCN | | | | 15 | T | T | NPE | NPPOC |
| 6 | dG^{tac} | iPr | EtCN | | | | 16 | dA^{tac} | T | EtCN | NPPOC |
| | | | | | | | 17 | dA^{Bz} | T | EtCN | NPPOC |
| | | | | | | | 18 | dC^{Ac} | T | EtCN | NPPOC |
| | | | | | | | 19 | dG^{tac} | T | EtCN | NPPOC |

chromatography (70 g silica gel, column 4×15 cm, and solvent gradient hexane/acetone (ratio 4:1) with 0,1% triethylamine to 1:1 with 0.1% triethylamine). The collected product fractions resulted in a yield of 6 Mol % after the solvent had been removed. The UV-spectrum in methanol showed characteristic bands at 301, 236 and 215 nm.

Examples 6 to 14 of oligonucleotides according to the invention synthesized by a process according to the invention are summarized in Table 1 and described in detail in the following:

Table 1: Starting Materials (1-10) and synthesized oligonucleotides (11-19)

Abbreviations in Table 1 represent the following groups:

Ac acetyl

Bz benzoyl

d deoxy

dmf dimethylaminomethylene

tac 1-(4-tert-butyl)-phenoxyacetyl

A, C, G, T represent adeninyl, cytosinyl, guaninyl, thyminyl respectively.

Compounds 1 to 19 as mentioned in table 1 are represented by the following formulae:

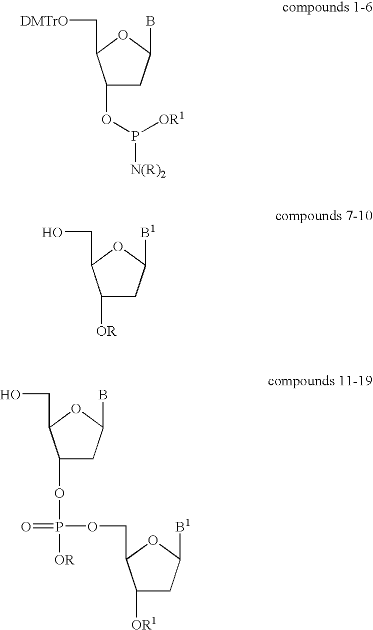

### Example 6

### Preparation of Thymidylyl-{3′-[O^{P}-(2-cyanoethyl)]→5′}-3′-O-[2-(2-nitrophenyl)propyloxycarbonyl]thymidine (11)

A mixture of 5′-O-(4,4′-dimethoxytrityl)thymidine-3′-O-[(2-cyanoethyl)-N,N-diisopropylphosphoramidite] (1) (4.3 g, 5.8 mmol), 3′-O-[2-(2-nitrophenyl)propyloxycarbonyl)thymidine (7) (2.0 g, 4.45 mmol) and 4,5-dicyanoimidazole (3.4 g, 29 mmol) in acetonitrile (50 ml) was stirred at room temperature (r.t.) under argon for 18 h and then treated with a solution of 0.5 M iodine in a mixture of dichloromethane/water/pyridine 1:1:3 (15 ml). After 20 min, the mixture was diluted with dichloromethane (400 ml), washed with saturated solution of sodium thiosulfate (2×100 ml) and then with water (1×100 ml). The organic layer was separated, dried over anhydrous sodium sulfate and evaporated. The rest was co-evaporated with toluene (2×50 ml) and treated with a solution of 2% toluene-4-sulfonic acid in a mixture of dichloromethane/methanol 4:1 (70 ml). After 10 min, the solution was diluted with dichloromethane (200 ml), washed with a saturated solution of sodium hydrogen carbonate (2×70 ml) and then with water (1×70 ml). The organic layer was separated, dried over anhydrous sodium sulfate and evaporated. The rest was dissolved in dichloromethane (50 ml), and the resulting solution added into n-hexane (500 ml). The precipitate was filtered off and purified by CC (silica gel, dichloromethane, dichloromethane/methanol 100:1 and then 20:1) to give 2.94 g (82%) of thymidylyl-{3′-[O^{P}-(2-cyanoethyl)]→5′}-3′-O-[2-(2-nitrophenyl)propyloxycarbonyl]thymidine (11) as a foam. UV (MeOH, λₘₐₓ nm (log ε): 263 (4.35), 210 (4.38). ¹H-NMR (DMSO-d₆, σ in ppm): 11.38 and 11.36 (2 s, 2 NH); 7.83 (d, H, ortho to NO₂); 7.71 (m, 2H arom. and H—C(6)); 7.51 (s, H-C(6)); 7.48 (m, H meta to NO₂); 6.16 (m, 2H—C(1′)); 5.25 (dd, CH₂OH); 5.10 and 4.98 (2m, 2H—C(3′)); 4.21 (m, H—C(4′), CNCH₂CH₂, POCH₂CH and COOCH₂); 4.06 (br.s, H—C(4′)); 3.58 (m, CH₂OH); 3.51 (m, CH₃CH); 2.92 (m, CNCH₂); 2.34 (m, 4H—C(2′)); 1.77 and 1.76 (2 s, 2Me-C(5)); 1.27 (d, CH₃CH).

### Example 7

### Preparation of Thymidylyl-{3′-[O^{P}-(2-cyanoethyl)]→540 }-3′-O-(4-nitrophenyloxycarbonyl)thymidine (12).

A mixture of 5′-O-(4,4′-dimethoxytrityl)thymidine-3′-O-[(2-cyanoethyl)-N,N-diisopropylphosphoramidite] (1) (0.83 g, 1.11 mmol), 3′-O-(4-nitrophenyloxycarbonyl)thymidine (8) (0.35 g, 0.85 mmol) and 4,5-dicyanoimidazole (0.65 g, 5.5 mmol) in acetonitrile (8 ml) was stirred at r.t. under argon for 4 h and then treated with a solution of iodine (0.4 g) in a mixture of dichloromethane/water/pyridine 1:1:3 (5 ml). After 20 min, the mixture was diluted with dichloromethane (80 ml), washed with saturated solution of sodium thiosulfate (2×30 ml) and then with phosphate buffer pH 7.0 (2×30 ml). The org. layer was separated, dried over anhydrous sodium sulfate and evaporated. The rest was co-evaporated with toluene (2×15 ml) and treated with a solution of 2% toluene-4-sulfonic acid in a mixture of dichloromethane/methanol 4:1 (5.2 ml). After 8 min, the solution was diluted with dichloromethane (80 ml), washed with a solution of sodium hydrogen carbonate (50 mg) in water (30 ml) and then with phosphate buffer pH7.0 (2×30 ml). The org. layer was separated, dried over anhydrous sodium sulfate and evaporated. The rest was purified by CC (silica gel, dichloromethane, dichloromethane/methanol 50:1 and then 9:1) to give 0.39 g (60%) of thymidylyl-{3′-[O^{P}-(2-cyanoethyl)]-5′}-3′-O-(4-nitrophenyloxycarbonyl)thymidine (12) as a foam. UV (MeOH, λₘₐₓ nm (log ε): 265 (4.46), 211 (4.41). 1H-NMR (DMSO-d₆, σ in ppm): 11.40 and 11.34 (2 s, 2NH); 8.32 (d, 2H, ortho to NO₂); 7.66 (s, H—C(6)); 7.60 (d, H meta to NO₂); 7.56 (s, H—C(6)); 6.22 (m, 2H—C(1′)); 5.31 (m, H—C(3′)); 5.22 (dd, CH₂OH; 4.99 (m, H—C(3′)); 4.45 (m, H—C(4′)); 4.34 (m, POCH₂CH); 4.22 (m, CNCH₂CH₂); 4.08 (br.s, H—C(4′)); 3.59 (m, CH₂OH); 2.93 (m, CNCH₂); 2.49 and 2.37 (2m, 4 H—C(2′)); 1.79 and 1.75 (2 s, 2 Me-C(5)).

### Example 8

### Preparation of Thymidylyl-{3′-[O^{P}-(2-cyanoethyl)]→5′}-N4-acetyl-2′-deoxy-3-O-[2-(2-nitrophenyl)propyloxycarbonyl]cytidine (13)

A mixture of 5′-O-(4,4′-dimethoxytrityl)thymidine-3′-O-[(2-cyanoethyl)-N,N-diisopropylphosphoramidite] (1) (3.66 g, 4.91 mmol), N⁴-acetyl-2′-deoxy-3′-O-[2-(2-nitrophenyl)propyloxycarbonyl)cytidine (9) (1.8 g, 3.78 mmol) and 4,5-dicyanoimidazole (2.23 g, 18.9 mmol) in acetonitrile (50 ml) was stirred at r.t. under nitrogen for 30 min and then treated with a solution of 0.5 M iodine in a mixture of dichloromethane/water/pyridine 1:1:3 (12 ml). After 20 min, the mixture was diluted with dichloromethane (500 ml), washed with saturated solution of sodium thiosulfate (2×150 ml) and then with water (1×150 ml). The organic layer was separated, dried over anhydrous sodium sulfate and evaporated. The rest was co-evaporated with toluene (2×50 ml) and treated with a solution of 2% toluene4-sulfonic acid in a mixture of dichloromethane/methanol 4:1 (63 ml). After 20 min, the solution was diluted with dichloromethane (500 ml), washed with a saturated solution of sodium hydrogen carbonate (2×150 ml) and then with water (1×150 ml). The organic layer was separated, dried over anhydrous sodium sulfate and evaporated. The rest was purified by CC (silica gel, ethylacetate, ethylacetate/methanol 100:1→20:1) to give 2 g (65%) of thymidylyl-{3′-[O^{P}-(2-cyanoethyl)]→5′}-N⁴-acetyl-2′-deoxy-3′-O-[2-(2-nitrophenyl)propyloxycarbonyl]cytidine (13) as a foam. UV (MeOH, λ nm (log ε): 300 sh (3.80), 252 (4.32), 212 (4.37). ¹H-NMR (DMSO-d₆, σ in ppm): 11.33 (s, NH)); 10.91 (s, NHAc); 8.10 (d, H—C(6) from Cyt); 7.84 (d, H ortho to NO₂); 7.71 (m, 2H arom. and H—C(6) from Thy); 7.48 (m, H meta to NO₂); 7.22 (d, H—C(5) from Cyt); 6.18 and 6.07 (2dd, 2H—C(1′)); 5.21 (dd, CH₂OH); 5.14 and 4.97 (2m, 2H—C(3′)); 4.29 (m, H—C(4′), POCH₂CH and COOCH₂); 4.20 (m, CNCH₂CH₂); 4.05 (br.s, H—C(4′)); 3.58 (m, CH₂OH); 3.50 (m, CH₃CH); 2.92 (m, CNCH₂); 2.35 (m, 4H—C(2′)); 2.08 (s, Ac), 1.76 (s, Me-C(5)); 1.27 (d, CH₃CH).

### Example 9

### Preparation of Thymidylyl-{3′-[O^{P}-(2-cyanoethyl)]→-5′}-2′-deoxy-N²-dimethylaminomethylene-3′-O-[2-(2-nitrophenyl)propyloxycarbonyl]guanosine (14)

A mixture of 5′-O-(4,4′-dimethoxytrityl)thymidine-3′-O-[(2-cyanoethyl)-N,N-diisopropylphosphoramidite] (1) (6.3 g, 8.5 mmol), 2′-deoxy-N²-dimethylaminomethylene-3′-O-[2-(2-nitrophenyl)propyloxycarbonyl)guanosine (10) (3 g, 5.66 mmol) and 4,5-dicyanoimidazole (5 g, 42.5 mmol) in acetonitrile (100 ml) was stirred at r.t. under nitrogen for 30 min and then treated with a solution of 0.5 M iodine in a mixture of dichloromethane/water/pyridine 1:1:3 (15 ml). After 20 min, the mixture was diluted with dichloromethane (500 ml), washed with saturated solution of sodium thiosulfate (2×200 ml) and then with water (1×200 ml). The organic layer was separated, dried over anhydrous sodium sulfate and evaporated. The rest was co-evaporated with toluene (2×50 ml) and treated with a solution of 2% toluene-4-sulfonic acid in a mixture of dichloromethane/methanol 4:1 (120 ml). After 20 min, the solution was diluted with dichloromethane (500 ml), washed with a saturated solution of sodium hydrogen carbonate (2×200 ml) and then with water (1×200 ml). The organic layer was separated, dried over anhydrous sodium sulfate and evaporated. The rest was purified by CC (silica gel, dichloromethane, dichloromethane/methanol 50:1-10:1) to give 2.24 g (45%) of thymidylyl-{3′-[O^{P}-(2-cyanoethyl)]→5′}-2′-deoxy-N⁴-dimethylaminomethylene-3′-O-[2-(2-nitrophenyl)propyloxycarbonyl]guanosine (14) as a foam. UV (MeOH, λₘₐₓ nm (log ε): 303 (4.35), 272 (4.32), 237 (4.30), 209 (4.42). ¹H-NMR (DMSO-d₆, σ in ppm): 11.35 (m, 2NH); 8.58 (s, CHN(Me)₂); 7.97 (s, H—C(8) from Gua); 7.83 (d, H ortho to NO₂); 7.73 (m, 2H arom. and H—C(6)); 7.51 (m, H meta to NO₂); 6.18 (m, 2H—C(1′)); 5.37 (m, CH₂OH); 5.18 and 4.94 (2 m, 2H—C(3′)); 4.34-3.98 (m, 2H—C(4′), CNCH₂CH₂, POCH₂CH and COOCH₂); 3.52 (m, CH₂OH and CH₃CH); 3.16 and 3.02 (2s, (Me)₂N); 2.88 (m, CNCH₂); 2.52 and 2.30 (2m, 4H—C(2′)); 1.76 (s, Me-C(5)); 1.28 (d, CH₃CH).

### Example 10

### Preparation of Thymidylyl-{3′-[O^{P}-2-(4-nitrophenyl)ethyl]→5′}-3′-O-[2-(2-nitrophenyl)propyloxycarbonyl]thymidine (15)

A mixture of 5′-O-(4,4′-dimethoxytrityl)thymidine-3′-O-[2-(4-nitrophenyl)ethyl-N,N-diethylphosphoramidite] (2) (5.6 g, 6.9 mmol), 3′-O-[2-(2-nitrophenyl)propyloxycarbonyl)thymidine (7) (2.1 g, 4.67 mmol) and 4,5-dicyanoimidazole (4.1 g, 34.7 mmol) in acetonitrile (80 ml) was stirred at r.t. under argon for 18 h and then treated with a solution of 0.5 M iodine in a mixture of dichloromethane/water/pyridine 1:1:3 (15 ml). After 30 min, the mixture was diluted with dichloromethane (500 ml), washed with saturated solution of sodium thiosulfate (2×100 ml) and then with water (2×100 ml). The organic layer was separated, dried over anhydrous sodium sulfate and evaporated. The rest was co-evaporated with toluene (2×50 ml) and treated with a solution of 2% toluene4-sulfonic acid in a mixture of dichloromethane/methanol 4:1 (100 ml). After 15 min, the solution was diluted with dichloromethane (400 ml), washed with a saturated solution of sodium hydrogen carbonate (2×100 ml) and then with water (1×100 ml). The organic layer was separated, dried over anhydrous sodium sulfate and evaporated. The rest was dissolved in dichloromethane (70 ml), and the resulting solution added into n-hexane (800 ml). The precipitate was filtered off and purified by CC (silica gel, dichloromethane, dichloromethane/methanol 100:1 and then 20:1) to give 2.15 g (51%) of thymidylyl-{3′-[O^{P}-2-(4-nitrophenyl)ethyl)]→5′}-3′-O-[2-(2-nitrophenyl)propyloxycarbonyl]thymidine (15) as a foam. UV (MeOH, λₘₐₓ nm (log ε): 264 (4.48), 210 (4.47). ¹H-NMR (DMSO-d₆, σ ppm): 11.37 and 11.36 (2s, 2NH); 8.14 and 8.12 (2d, 2H ortho to NO₂ from NPE); 7.81 (d, H ortho to NO₂ from NPPOC); 7.70-7.44 (m, 5H, arom. and 2H—C(6)); 6.12 (m, 2H—C(1′)); 5.20 (br.s CH₂OH); 5.05 and 4.85 (2m, 2H—C(3′)); 4.31-3.93 (m, 2H—C(4′), 2×(POCH₂) and COOCH₂); 3.51 (m, CH₂OH); 3.44 (m, CH₃CH); 3.07 (m, POCH₂CH₂); 2.27 (m, 4H—C(2′)); 1.75 and 1.72 (2s, 2Me-C(5)); 1.27 (d, CH₃CH).

### Example 11

### Preparation of N⁶-[1-(4-tert-Butyl)phenyloxyacetyl)]-2′-deoxyadenylyl-{3′-[O^{P}-(2-cyanoethyl)]→5′}-3′-O-[2-(2-nitrophenyl)propyloxycarbonyl)thymidine (16)

A mixture of N⁶-[1-(4-tert-butyl)phenyloxyacetyl)]-2′-deoxy-5′-O-(4,4′-dimethoxytrityl)adenosine-3′-O-[(2-cyanoethyl)-N,N-diisopropylphosphoramidite] (3) (0.94 g, 1 mmol), 3′-O-[2-(2-nitrophenyl)propyloxycarbonyl)thymidine (7) (0.36 g, 0.8 mmol) and 4,5-dicyanoimidazole (0.59 g, 5 mmol) in acetonitrile (10 ml) was stirred at r.t. under nitrogen for 1 h and then treated with a solution of 0.5 M iodine in a mixture of dichloromethane/water/pyridine 1:1:3 (2 ml). After 20 min, the mixture was diluted with dichloromethane (100 ml), washed with saturated solution of sodium thiosulfate (2×30 ml) and then with water (1×30 ml). The organic layer was separated, dried over anhydrous sodium sulfate and evaporated. The rest was co-evaporated with toluene (2×15 ml) and treated with a solution of 2% toluene4-sulfonic acid in a mixture of dichloromethane/methanol 4:1 (25 ml). After 15 min, the solution was diluted with dichloromethane (100 ml), washed with a saturated solution of sodium hydrogen carbonate (2×30 ml) and then with water (1×30 ml). The organic layer was separated, dried over anhydrous sodium sulfate and evaporated. The rest was purified by CC (silica gel, dichloromethane and then with dichloromethane/methanol 50:1→10:1) to give 0.48 g (60%) of N⁶-[1-(4-tert-butyl)phenyloxyacetyl)]-2′-deoxyadenylyl-{3′-[O^{P}-(2-cyanoethyl)]-5′}-3′-O-[2-(2-nitrophenyl)propyloxycarbonyl)thymidine (16) as a foam. UV (MeOH, λₘₐₓ nm (log ε): 271 (4.42), 215 (4.45). ¹H-NMR (DMSO-d₆, σ in ppm): 11.39 and 11.36 (2s, NH from Thy, diastereoisomers); 10.92 (s, NH from Ade); 8.70, 8.69, 8.67 and 8.65 (4s, 2H—C(2) and 2H—C(8) from Ade, diastereoisomers); 7.82 (d, H ortho to NO₂); 7.68 (m, 2H from NO₂Ph); 7.53 (s, H—C(6)); 7.47 (m, H meta to NO₂); 7.29 (d, 2H meta to t-Bu); 6.87 (d, 2H ortho to t-Bu); 6.49 and 6.14 (2m, 2H—C(1′)); 5.16 (m, 2H—C(3) and CH₂OH); 4.99 (s, NHCOCH₂); 4.27 (m, 2H—C(4′), CNCH₂CH₂, POCH₂CH and COOCH₂); 3.60-3.10 (m, CH₂OH, CH₃CH, and H—C(2′)); 2.95 (m, CNCH₂); 2.70 (m, H—C(2′); 2.33 (m, 2H—C(2′)); 1.78 and 1.76 (2s, 2Me-C(5)); 1.26 (d, CH₃CH); 1.23 (s, 9H from t-Bu).

### Example 12

### Preparation of N⁶-Benzoyl-2′-deoxyadenylyl-{3′-[O^{P}-(2-cyanoethyl)]→5′}-3′-O-[2-(2-nitrophenyl)propyloxycarbonyl)thymidin (17)

A mixture of N⁶-bensoyl-2′-deoxy-5′-O-(4,4′-dimethoxytrityl)adenosine-3′-O-[(2-cyanoethyl)-N,N-diisopropylphosphoramidite] (4) (4.58 g, 5.33 mmol), 3′-O-[2-(2-nitrophenyl)propyloxycarbonyl)thymidine (7) (1.4 g, 3.11 mmol) and 4,5-dicyanoimidazole (3.19 g, 26.7 mmol) in acetonitrile (100 ml) was stirred at r.t. under nitrogen for 30 min and then treated with a solution of 0.5 M iodine in a mixture of dichloromethane/water/pyridine 1:1:3 (18 ml). After 20 min, the mixture was diluted with dichloromethane (600 ml), washed with saturated solution of sodium thiosulfate (2×250 ml) and then with water (1×250 ml). The organic layer was separated, dried over anhydrous sodium sulfate and evaporated. The rest was co-evaporated with toluene (2×50 ml) and treated with a solution of 2% toluene4-sulfonic acid in a mixture of dichloromethane/methanol 4:1 (170 ml). After 20 min, the solution was diluted with dichloromethane (600 ml), washed with a saturated solution of sodium hydrogen carbonate (2×200 ml) and then with water (1×200 ml). The organic layer was separated, dried over anhydrous sodium sulfate and evaporated. The rest was purified by CC (silica gel, ethylacetate, ethylacetate/methanol 100:1-10:1) to give 1.98 g (69%) of N⁶-bensoyl-2′-deoxyadenylyl-{3′-[O^{P}-(2-cyanoethyl)]→5′}-3′-O-[2-(2-nitrophenyl)propyloxycarbonyl)thymidine (17) as a foam. UV (MeOH, λₘₐₓ nm (log ε): 274 (4.41), 212 (4.38). ¹H-NMR (DMSO-d₆, C in ppm): 11.38 and 11.36 (2s, NH from Thy, diastereoisomers); 11.22 (s, NH from Ade); 8.74, 8.73, 8.68 and 8.67 (4 s, 2H—C(2) and 2H—C(8) from Ade, diastereoisomers); 8.03 (d, 2H ortho from Bz); 7.70-7.44 (m, 6H arom. and H—C(6)); 6.52 and 6.13 (2m, 2H—C(1′)); 5.22 (m, H—C(3′) and CH₂OH); 5.14 (m, H—C(3′)); 4.29 (m, 2H—C(4′), CNCH₂CH₂, POCH₂CH and COOCH₂); 3.60 (m, CH₂OH); (m, CH₃CH); 3.08 (m, H—C(2′)); 2.96 (m, CNCH₂); 2.68 (m, H—C(2′); 2.33 (m, 2H—C(2′)); 1.79 and 1.77 (2s, Me-C(5), diastereoisomers); 1.26 and 1.25 (2d, CH₃CH, diastereoisomers).

### Example 13

Preparation of N⁴-Acetyl-2′-deoxycytidylyl-{3′-[O^{P}-(2-cyanoethyl)]→5′}-3′-O-[2-(2-nitrophenyl)propyloxycarbonyl)thymidine (18)

A mixture of N⁴-acetyl-2′-deoxy-5′-O-(4,4′-dimethoxytrityl)cytidine-3′-O-[(2-cyanoethyl)-N,N-diisopropylphosphoramidite] (5) (5.58 g, 7.22 mmol), 3′-O-[2-(2-nitrophenyl)propyloxycarbonyl)thymidine (7) (2.5 g, 5.56mmol) and 4,5-dicyanoimidazole (3.28 g, 27.8mmol) in acetonitrile (100ml) was stirred at r.t. under nitrogen for 30min and then treated with a solution of 0.5 M iodine in a mixture of dichloromethane/water/pyridine 1:1:3 (18ml). After 20min, the mixture was diluted with dichloromethane (600ml), washed with saturated solution of sodium thiosulfate (2×250ml) and then with water (1×250ml). The organic layer was separated, dried over anhydrous sodium sulfate and evaporated. The rest was co-evaporated with toluene (2×50ml) and treated with a solution of 2% toluene-4-sulfonic acid in a mixture of dichloromethane/methanol 4:1 (170ml). After 20min, the solution was diluted with dichloromethane (600ml), washed with a saturated solution of sodium hydrogen carbonate (2×200ml) and then with water (1×200ml). The organic layer was separated, dried over anhydrous sodium sulfate and evaporated. The rest was purified by CC (silica gel, ethylacetate, ethylacetate/methanol 100:1-10:1) to give 2.92 g (63%) of N⁴-acetyl-2′-deoxycytidylyl-{3′-[O^{P}-(2-cyanoethyl)]→5′}-3′-O-[2-(2-nitrophenyl)propyloxycarbonyl)thymidine (18) as a foam. UV (MeOH, λₘₐₓ nm (log ε): 300 sh (3.87), 250 (4.35), 212 (4.39). ¹H-NMR (DMSO-d₆, σ in ppm): 11.36 (s, NH)); 10.90 (s, NHAc); 8.24 (d, H—C(6) from Cyt); 7.85 (d, H ortho to NO₂); 7.70 (m, 2H arom.); 7.51 (s, H—C(6) from Thy); 7.49 (m, H meta to NO₂); 7.21 (d, H—C(5) from Cyt); 6.13 (m, 2H—C(1′)); 5.20 (dd, CH₂OH); 5.10 and 4.98 (2m, 2H—C(3′)); 4.26 (m, 2H—C(4′), POCH₂CH, CNCH₂CH₂, and COOCH₂); 3.60 (m, CH₂OH); 3.50 (m, CH₃CH); 2.93 (m, CNCH₂); 2.60 (m, H—C(2′)); 2.30 (m, 3H—C(2′)); 2.08 (s, Ac), 1.77 (s, Me-C(5)); 1.27 (d, CH₃CH).

### Example 14

### Preparation of N²-[ -(4-tert-Butyl)phenyloxyacetyl)]-2′-deoxyguanylyl-{3′-[O^{P}-(2-cyanoethyl)]→5′}-3′-O-[2-(2-nitrophenyl)propyloxycarbonyl)thymidine (19)

A mixture of N²-[1-(4-tert-butyl)phenyloxyacetyl)]-2′-deoxy-5′-O-(4,4′-dimethoxytrityl)guanosine-3′-O-[(2-cyanoethyl)-N,N-diisopropylphosphoramidite] (6) (7.26 g, 7.56mmol), 3′-O-[2-(2-nitrophenyl)propyloxycarbonyl)thymidine (7) (2 g, 4.45 mmol) and 4,5-dicyanoimidazole (4.47 g, 37.85mmol) in acetonitrile (100ml) was stirred at r.t. under nitrogen for 3 h and then treated with a solution of 0.5 M iodine in a mixture of dichloromethane/water/pyridine 1:1:3 (20ml). After 20min, the mixture was diluted with dichloromethane (500ml), washed with saturated solution of sodium thiosulfate (2×250ml) and then with water (1×250ml). The organic layer was separated, dried over anhydrous sodium sulfate and evaporated. The rest was co-evaporated with toluene (2×50ml) and treated with a solution of 2% toluene-4-sulfonic acid in a mixture of dichloromethane/methanol 4:1 (120ml). After 15min, the solution was diluted with dichloromethane (500ml), washed with a saturated solution of sodium hydrogen carbonate (2×200ml) and then with water (1×200ml). The organic layer was separated, dried over anhydrous sodium sulfate and evaporated. The rest was purified by CC (silica gel, ethylacetate, ethylacetate/methanol 50:1 10: land then with dichloromethane/methanol 9:1) to give 2.2 g (48%) of N²-[1-(4-tert-butyl)phenyloxyacetyl)]-2′-deoxyguanylyl-{3′-[O^{P}-(2-cyanoethyl)]→5′}-3′-O-[2-(2-nitrophenyl)propyloxycarbonyl)thymidine (19) as a foam. UV (MeOH, λ nm (log ε): 277 sh (4.25), 260 (4.38), 210 (4.44). hu 1H-NMR (DMSO-d₆, σ in ppm): 11.80 (s, H—N(1) and NH—C(2) from Gua); 11.39 (s, NH from Thy); 8.27 (s, H—C(8) from Gua); 7.80 (m, H ortho to NO₂); 7.66 (m, 2H arom. from NO₂Ph); and)); 7.48 (m, H—C(6) and H meta to NO₂); 7.30 (d, 2H meta to t-Bu); 6.87 (d, 2H ortho to t-Bu); 6.23 (m, 2H—C(1′)); 5.16 (m, CH₂OH); 5.15 and 5.10 (2m, 2H—C(3′)); 4.82 (s, NHCOCH₂); 4.27 (m, 2H—C(4′), CNCH₂CH₂, POCH₂CH and COOCH₂); 3.58 (m, CH₂OH); 3.46 (m, CH₃CH); 2.95 (m, CNCH₂ and H—C(2′)); 2.67 (m, H—C(2′)); 2.41 (m, 2H—C(2′)); 1.70 (s, Me-C(5)); 1.28 (m, CH₃CH and 9H from t-Bu).

### Example 15

### General procedure (A) for the synthesis of 5′-0-Dimethoxytrityl-3′-0-(9-fluorenylmethoxycarbonyl)-2′-deoxynucleosides (20, 21)

A solution of 420mg (1.6mmol) (9-fluorenylmethyl)chloroformiat in 4ml anhydrous dichloromethanewas dropped to 1 of mmol 5′-0-dimethoxytrityl-2′-deoxy-nucleoside (coevapurated with pyridine in 4ml anhydrous pyridine. After stirring 4 h at r.t. the mixture was diluted with 15ml H₂O and extracted with CH₂Cl₂. The organic phase was dried out MgSO₄, filtered, and evaporated. The crude product was purified by CC (silica gel, PE/EE 3:1 to 1:2) to give the desired products (20, 21).

### Example 16

### Preparation of 5′-0-Dimethoxytrityl-3′-0-(9-fluorenylmethoxycarbonyl)-thymidine (20)

Compound 20 was prepared in 89% yiel following the general procedure A using 3.57 g (13.8mmol) (p-fluorenylmethyl)chloroformiat/15ml anhydrous dichloromethane and 4.42 g (8.1 mmol) 5′-0-dimethoxytritylthymidine/15ml anhydrous pyridine. UV (MeOH), λ [nm]: 204 (4.99), [216 (4.62)]. [234 (4.39)], [255 (4.42)]. 263 (4.48), [268 (4.41)], [285 (3.90)]. [298 (3.75)]; ¹H-NMR (250 MHz, CDCl₃(s, 1H, NH). 7.75 (d, 2H, 2×arom. H FMOC), 7.59 (m, 3H, 2×arom. H FMOC, H—C(6)), 7.43-7.23 (m, 13H. 4×arom. H FMOC, 9×arom. H DMTr), 6.81 (d, 4H, 4×arom. H DMTr), 6.47 (m, 1H, H—C(1′)), 5.34 (m. 1H, H—C(3′)), 4.41 (m, 2H, CHa FMOC), 4.23 (m. 2H, H—C(4′), H—C(9) FMOC), 3.76 (s, 6H, 2×OCH₃ DMTr), 3.47 (m, 2H. 2×H—C(5′)), 2.51-2.42 (m, 2H, 2×H—C(2′)), 1.36 (s, 3H CH₃ Thy); Anal. calcd. for C₄₆H₄₂N₂O₉×0.5H2O (775.87). C 71.21, H 5.59, N 3.61; found: C 71.07, H 5.74, N 3.55.

### Example 17

### Preparation of N4-Benzoyl-5′-O-dimethoxytrityl-3′-O-(9-fluorenylmethoxy-carbonyl)-2′-deoxycytidine (21)

Compound 21 was prepared in 87% yield following the general procedure A using 4.4 g (17mmol) (9-fluorenylmethyl)chloroformiat/20ml anhydrous dichloromethane and 6.34 g (10mmol) N4-benzoyl-5′-O-dimethoxytrityl-2′-deoxycytidine/20ml anhydrous pyridine.

UV (MeOH), λₘₐₓ [nm]: 204 (5.03). [216 (4.69)], 236 (4.55), 260 (4.62), [268 (4.52)], 298 (4.19), [309 (4.01)]; ¹H-NMR (250 MHz, CDCl₃): 8.79 (s, 1H, NH), 8.15 (d, 1H, H—C(5)), 7.88 (d, 2H. 2×arom. H FMOC), 7.75 (d, 2H, 2×arom. H Bz), 7.60 (d, 2H, 2×arom. H FMOC), 7.61-7.21 (m, 17H, 4×arom. H FMOC, H—C(6), 9×arom. H DMTr, 3×arom. H Bz), 6.83 (dd, 4H, 4×arom. H DMTr), 6.34 (m, 1H, H—C(1′)). 5.31 (m, 1H, H—C(3′)), 4.43 (m, 2H, CH₂ FMOC), 4.35 (m, 1H, H C(4′)), 4.25 (t, 1H, H—C(9) FMOC), 3.76+3.75 (2×s, 6H, 2×OCH₃ DMTr), 3.48 (m, 2H, 2×H—C(5′)), 2.91 (m, 1H. H—C(2′)), 2.34 (m, 1H, H—C(2′)); Anal. calcd. for C₅₂H₄₇N₃O₉×0.5H2O (866.98). C 72.04, H 5.58, N 4.84; found: C 71.62. H 5.43, N 4.80.

### Example 18

### General procedure (B) for the synthesis of 3′-0-(9-Fluorenylmethoxycarbonyl)-2′-deoxynucleosides (22, 23)

1mmol 5′-0-Dimethoxytrityl-3′-0-(9-fluorenylmethoxycarbonyl)-2′-deoxynucleoside (20, 21) was dissolved in 10ml of a 2% solution of toluene4-sulfonic acid in dichloromethane/methanol 4:1. After stirring at r.t. for 1 h the mixture was diluted with 15 ml H2O, and extracted twice with CH₂Cl₂. The organic phase was dried over MgSO₄, filtered, and evaporated. The crude product was purified by CC (silica gel, CH₂Cl₂/MeOH, 100:0 to 100:3.5) to give the desired products (22, 23).

### Example 19

### Preparation of3′-0-(9-Fluorenylmethoxycarbonyl)-thymidine (22)

Compound 22 was prepared in 95% yield following the general procedure B using 450mg (0.59mmol) 5′-0-dimethoxytrityl-3′-0-(9-fluorenylmethoxycarbonyl)-thymidine (1)/5.9ml of 2% toluene-4-sulfonic acid-solution. UV (MeOH), λmax [nm]: 206 (4.72), [218 (4.35)], [224 (4.02)]. [256 (4.40)]2623 (4.46), [268 (4.39)], [285 (3.83)], 298 (3.73); ¹H-NMR (250 MHz, CDCl₃): 8.27 (s, 1H, NH), 7.76 (d. 2H, 2×arom. H FMOC), 7.59 (d, 2H, 2×arom. H FMOC). 7.44-7.26 (m, 5H, 4×arom. H FMOC, H—C(6)), 6.19 (m, 1H. H—C(1′)). 5.26 (m, 1H, H—C(3′)), 4.45 (d, 2H, CH₂ FMOC), 4.24 (t, 1H, H—C(9) FMOC), 4.15 (m, 1H, H—C(4′)), 3.88 (m, 2H. 2×H—C(5′)), 2.47 (m, 3H, OH—C(5′), 2×H—C(2′)), 1.91 (s, 3H, CH₃ Thy); Anal. calcd. for C₂₅H₂₄N₂O_{7 ×0.5}H₂O (473.49). C 63.42, H 532, N 5.92; found: C 63.38, H 5.24. N 5.81.

### Example 20

### Preparation of N⁴-Benzoyl-3′-O-(9-fluorenylmethoxycarbonyl)-2′-deoxycytidine (23)

Compound 23 was prepared in 78% yield following the general procedure B using 7.23 g (8.4mmol) N⁴-benzoyl-5′-O-dimethoxytrityl-3′-O-(9-fluorenylmethoxycarbonyl)-2′-deoxycytidine (2)/84 ml of 2% toluene4-sulfonic acid-solution.

UV (MeOH), λₘₐₓ [nm]: 205 (4.80), [216 (4.49)], [224 (4.23)], [256 (4.61)]. 260 (4.63), [268 (4.52)], [288 (4.13)], 298 (4.20), [306 (4.04)]; ¹H-NMR (250 MHz, DMSO-d₆): 11.27 (s(br), 1H, NH), 8.34 (d, 1H, H—C(5)), 7.99 (d, 2H, 2×arom. H FMOC), 7.90 (d, 2H, 2×arom. H Bz), 7.68-7.32 (m, 10H, 6×arom. H FMOC, H—C(6), 3×arom. H Bz), 6.10 (m, 1H, H—C(1′)), 5.21 (t, 1H, OH—C(5′)), 5.11 (m, 1H, H—C(3′)), 4.60 (d, 2H, CH₂ FMOC), 4.33 (t, 1H, H—C(9) FMOC), 4.11 (m, 1H, H—C(4′)), 3.62 (m. 2H, 2×H—C(5′)), 2.43 (m, 1H, H—C(2′)), 2.26 (m, 1H, H—C(2′)); Anal. calcd. for C₃₁H₂₇N₃0₇ ×0.5H2O (553.58). C 67.26, H 4.92, N 7.59; found: C 66.83, H 4.83, N 7.51.

### Example 21

### General procedure (C) for the synthesis of5′-O-Dimethoxytrityl-2′deoxynucleoside-{3′(O^{P}cyanoethyl)-5′}-3′-O-(9-fluorenyl-methoxycarbonyl)-2′-deoxynucleoside (24, 25)

To a solution of 1 mmol 3′-0-(9-fluorenylmethoxycarbonyl)-2′-deoxynucleoside (22, 23) and 1.7 mmol 5′-0-dimethoxytrityl-2′-deoxynucleosid-3′-0-[(2-cyanoethyl)(N,N-diisopropylamino)]phosphitamide in 15 ml anhydrous acetonitrile was added under argaon atmosphere 4.4mmol of 1H tetrazole. After stirring for 3 h at r.t. was added a mixture of oxidizing solution (500 mg iodinein 5 ml pyridine/water/dichloromethane 3/1/1) until iodine colour persisted. After 1 h the mixture was diluted with 40 ml dichloromethane, and extracted twice with 40 ml of a saturated solution of sodium thiosulfate. The aqueous washings were combined and re-extracted with 40 ml dichloromethane. The organic phase was dried over MgSO₄, filtered and evaporated. The crude product was purified by CC (silica gel CH₂Cl₂/MeOH, 100:0 to 100:3.5) to give the desired products (24, 25).

### Example 22

### Preparation of N⁶-Benzoyl-5′-O-dimethoxytrityl-2′-deoxyadenylyl-{3′-(O^{P}-cyanothyl)-5′-}3′-0-(9-fluorenylmethoxycarbonyl)-thymidine (24)

Compound 24 was prepared in 90% yield following the general procedure C using 465 mg (1 mmol) 3′-0-(9-fluorenylmethoxycarbonyl)-thymidine (22), 1.4 g (1.7 mmol) N⁶-benzoyl-5′-O-dimethoxytrityl-2′-deoxyadenosine-3′-0-[(2-cyanoethyl)(N,N-diisopropylammo)]phosphitamide, 280 mg (4 mmol) tetrazole/15 ml anhydrous acetonitrile.

UV (MeOH), λₘₐₓ [nm]: 204 (5.09), [217 (4.77)], [224 (4.64)], [233 (4.59), 264 (4.61), [271 (4.60)], [284 (4.42)], [295 (4.15)], [320 (3.32)]; ¹H-NMR (250 MHz, CDCl₃): 9.12+9.08+8.86 (3×s, 2H. NH T/dA), 8.68+8.12 (2×2s, 2H, H—C(2), H—C(8) dA). 8.01 (d, 2H, 2×arom. H FMOC), 7.74 (d, 2H, 2×arom. H FMOC). 7.61-7.16 (m, 19H, H—C(6) T, 4×arom. H FMOC, 9×arom. H DMTr, 5×arom. H Bz), 6.78 (m. 4H. 4×arom. H DMTr), 6.48 (m, 1H, H—C(1′)), 6.25 (m, 1H, H—C(1′)), 5.29 (m. 1H, H—C(3′)), 5.24 (m, 1H, H—C(3′)), 4.444.19 (m, 9H, CH₂ FMOC, 2×H—C(5′), H—C(9) FMOC, 2×H—C(4′), α-CH₂ CE), 3.74+3.73 (2×s, 6H, 2×OCH₃ DMTr), 3.42 (m, 2H, 2×H—C(5′)), 3.12 (m. 1H, H—C(2′)) 2.82-2.61 (m, 3H, H—C(2′), β—CH₂ CE), 2.58-2.29 (m, 2H, 2×H—C(2′)), 1.89 (2×s, 3H, CH₃ T); ³¹P-NMR (400 MHz, CDCl³): 1.12.

### Example 23

### Preparation of 5′-O-Dimethoxytrityl-thymidylyl-{3′-(O^{P}-cyanoethyl)5′-}-N4-benzoyl-3′-O-(9-fluorenylmethoxycarbonyl)-2′-deoxycytidine (25)

Compound 25 was prepared in 96% yield following the general procedure C using 1.38 g (2.5 mmol) N4-benzoyl-3′-0-(9-fluorenylmethoxycarbonyl)-2′-deoxycytidine (23), 3.02 g (4.25 mmol) 5′-0-dimethoxytrityl-thymidine-3′-0-[(2-cyanoethyl)-(N,N-diisopropylamino)]phosphitamide, 790 mg (11.25 mmol) tetrazole/30 ml anhydrous acetonitrile.

UV (MeOH), λₘₐₓ [nm]: 204 (5.08), [217 (4.73)], [236 (4.57)], 262 (4.68),[282 (4.46)], 298 (4.15), [305 (3.99)]; ¹H-NMR (250 MHz, CDCl₃): 8.85 (s, 1H, NH T oder dC), 8.51+8.47 (2×s(br). 1H, NH T oder dC), 8.03 (2×d. 1H, H—C(5) dC), 7.87 (m. 2H, 2×arom. H FMOC), 7.75 (d, 2H, 2×arom. H FMOC), 7.62-7.17 (m, 20H, H—C(6) T. H—C(6) dC, 4×arom. H FMOC, 9×arom. H DMTr, 5×arom. H Bz), 6.80 (m, 4H, 4×arom. H DMTr), 6.36 (m, 1H, H—C(1′)), 6.28 (m, 1H, H—C(1′)), 5.18 (m, 2H. 2×H—C(3′)), 4.414.12 (m, 9H, CH₂ FMOC, 2×H—C (5′), H—C(9) FMOC, 2×H—C(4′), α-CH₂ CE), 3.75+3.74 (2×s, 6H, 2×OCH₃ DMTr), 3.51 (m, 1H, H—C(5′)), 3.37 (m, 1H, H—C(5′)), 2.85 (m, 1H, H—C(2′)), 2.73 (m, 1H, H—C(2′)), 2.63 (m, 2H, O—CH₂ CE). 2.44 (m, 1H, H—C(2′)), 2.20 (m, 1H, H—C(2′)), 1.39+1.38 (2×s, 3H, CH₃ T); ³¹P-NMR (400 MHz, CDl₃): 1.46+1.32; Anal. calcd. for C₆₅H₆₁N₆O₁₆P×H2O (1231.23). C 63.41, H 5.16. N 6.83; found: C 63.28, H 5.16, N 6.63.

### Example 24

### General procedure (D) for the synthesis of2′-Deoxynucleoside-{3′-(O^{P}-cyanoethyl) -5′-}-3′-0-(9-fluorenylmethoxy-carbonyl)-2′-deoxynucleoside (26, 27)

1 mmol 5′-Dimethoxytrityl-2′-deoxynucleoside-{3′-(O^{P}-cyanoethyl)-5′-3′-O-(9-fluorenylmethoxycarbonyl)-2′-deoxynucleoside (24, 25) was dissolved in 10 ml of a 2% solution of toluene4-sulfonic acid in dichloromethane/methanol 4:1. After stirring at r.t. for 30 min the mixture was diluted with 15 ml HaO, and extracted twice with CHaCla. The organic phase was dried over MgSO4, filtered, and evaporated. The crude product was purified by CC (silica gel, CHzCla/MeOH, 100:0 to 100:5) to give the desired products (26, 27).

### Example 25

### Preparation of N6-Benzoyl-2′-deoxyadenylyl-3′-(OP-cyanoethyl)-5′}-3′O-(9-fluorenyl-methoxycarbonyl)-thymidine (26)

Compound 26 was prepared in 79% yield following the general procedure D using 1.87 g (1.5 mmol) N6-benzoyl-5′-O-dimethoxytrityl-2′-deoxyadenylyl-{3′-(O^{P}-cyanoethyl)-5′-}-3′-0-(9-fluorenylmethoxycarbonyl)-thymidine (24)/15 ml 2% toluene-4-sulfonic acid-solution.

UV (MeOH), λₘₐₓ [nm]: 205 (4.87), [217 (4.57)], [225 (4.34)], 264 (4.60), [270 (4.58)], [283 (4.41)], [295 (4.14)], [322 (3.08)]; ¹H-NMR (250 MHz, CDCI₃): 9.82+9.72+9.43 (3×s, 1H, NH T, dA), 8.75+8.15 (2×2s, 2H. H—C(2), H—C(8) dA), 7.99 (d, 2H, 2×arom. H FMOC), 7.74 (d, 2H, 2×arom. H FMOC), 7.59-7.25 (m, 1OH, H—C(6) T, 4×arom. H FMOC, 5×arom. H Bz). 6.37 (m. 1H, H—C(1′)), 6.21 (m, 1H, H—C(1′)). 5.92 (d(br). 1H, OH—C(5′)), 5.33 (m. 1H, H—C(3′)), 5.22 (m, 1H, H—C(3′)). 4.464.20 (m, 9H, CH₂ FMOC, 2×H—C(5′), H—C(9) FMOC, 2×H—C(4′) α-CH₂ CE), 3.87 (m, 2H, 2×H—C(5′)), 3.14 (m, 1H, H—C(2′)), 2.77 (m, 2H, /3-CHa CE), 2.62 (m, 1H, H—C(2′)), 2.41 (m, 2H, 2×H—C(2′)), 1.89+1.88 (2×s, 3H, CH3 T); ³¹P-M (400 MHz, CDCl₃): 1.15+0.93; Anal. calcd. for C₄₅H₄₃N₈O₁₃P×2H₂O (970898). C 55.67, H 4.88, N 11.54; found: C 55.37, H 4.67, N 11.27.

### Example 26

### Preparation of Thymidylyl-{3′-(O^{P}-cyanoethyl-5′-}-N4-benzoyl-3′-O-(9-fluorenyl-methoxycarbonyl)-2′-deoxycytidine (27)

Compound 27 was prepared in 84% yield following the general procedure D using 1 g (0.82mmol) 5′-0-dimethoxytrityl-thymidylyl-{3′-(O^{P}-cyanoethyl)-5′}-N4-benzoyl-3′-0-(9-fluorenylmethoxycarbonyl)-2′-deoxycytidine (25)/9 ml 2% toluene-4-sulfonic acid-solution.

UV (MeOH), λₘₐₓ [nm]: 206 (4.82), [217 (4.54)], [223 (4.32)], 261 (4.69), [269 (4.60)], [286 (4.18)], 298 (4.17), [307 (4.00)]; ¹H-NMR (250 MHz, CDCl₃): 9.15+8.87+8.84 (3×s. 1H, NH T, dC), 8.08 (m, 1H, H—C(5) dC), 7.90 (dd, 2H, 2 ×arom. H FMOC), 7.75 (d, 2H, 2×arom. H FMOC), 7.63-7.29 (m, 1H, H—C(6) T, H—C(6) dC. 4×arom. H FMOC, 5×arom. H Bz), 6.27 (m, 1H, H—C(1′)), 6.11 (m, 1H. H—C(1′)), 5.24 (m, 2H, 2×H—C(3′)), 4.44-4.20 (m, 9H, CH₂ FMOC, 2 ×H—C(5′), H—C(9) FMOC, 2×H—C(4′), α-CH₂ CE). 3.85 (m. 2H, 2×H—C (5′)), 3.32 (s(br), 1H, OH—C(5′)), 2.80 (m. 3H, H—C(2′), β-CH2 CE), 2.53 (m, 2H, 2×H—C(2′)), 2.31 (m, 1H, H—C(2′)), 1.88 (s, 3H, CH₃ T) ³¹P-NMR (400 MHz, CDCl₃): 1.44+1.39; Anal. calcd. for C₄₄H₄₃N₆O₁₄P×2H2O (946878). C 55.81, H 5.00, N 8.88; found: C 55.45, H 4.76, N 8.84.

1. process for the preparation of polynucleotides, comprising the following steps:
a) reaction of the free 5′-hydroxy group of a selected oligonucleotide, whose terminal 3′-hydroxy group contains a usual suitable protecting group, derivatized in a previous step to a phosphite amidoester, phosphotriester or phosphonic acid ester, which is a 3′-hydroxy group of a free or solid phase bound polynucleotide or a solid phase bound hydroxy group, under suitable conditions and purification of the reaction product if necessary;
b) if necessary oxidation of the reaction product according step a) to a phosphodiester or phosphotriester, if a hydroxy group derivatized to a phosphite amidoester—was used and purification of the reaction product if necessary;
c) removal of the 3′-hydroxy protecting group of the reaction product according steps a) or b) under usual suitable conditions and purification of the reaction product if necessary;
d) derivatization of the free 3′-hydroxy group to a phosphite amidoester. phosphotriester or phosphonic acid ester by using usual suitable reagents;
e) if necessary rerun of steps a) to c) by using the activated reaction product according to step d),
whereby the oligonucleotides with the free 5′-hydroxy group according to step a) are always selected in such way, that the desired polynucleotide is obtained.

2. Process according to claim 1, comprising steps a) to c), characterized in that the 5′-hydroxy group of the selected oligonucleotide is a phosphite amidoester or phosphonic acid ester and is reacted with the free 3′-hydroxy group of a free or solid phase bound polynucleotide or with the hydroxy group of a solid phase in step a).

3. Process according to claim 1 or 2, characterized in that the selected oligonucleotide is at least one of a pentanucleotide, a tetranucleotide, a trinucleotide and a dinucleotide.

4. Process according to claim 1 or 2, characterized in that the protecting group of the 3′-hydroxy group of the selected oligonucleotide is a photolabile protecting group.

5. Process according to claim 1 or 2, characterized in that in addition to the selected oligonucleotides, also selected and correspondingly derivatized mononucleosides are used.

6. Process according to claim 1 or 2, characterized in that the compounds, which have a hydroxy group derivatized as phosphite amidoester, phosphotriester or phosphonic acid ester, are solid phase bound, whereby the solid phase is at least one of silica gel, glass, metal, preferably magnetic metal, plastic, cellulose, dextrane crosslinked with epichlorohydrine, agarose, styrene-divinylbenzene resin, and chloromethylated co-polystyrene-divinylbenzene resin.

7. Process according to claim 6, characterized in that the nucleotides are covalently bound to the solid phase via linker molecules.

8. Process according to claim 1 or 2, characterized in that the polynucleotides are DNA- or RNA-nucleotides or polynucleotides made from nucleic acid analogs.

9. Process according claim 1 or 2, characterized in that the steps are performed within an automated process.

10. Process according to claim 9, characterized in that the automated process is designed as parallel synthesis to the creation of a nucleotide library, where the selected oligonucleotides are selected specifically or at random.

11. Nucleotide derivative according to the general formula (L) 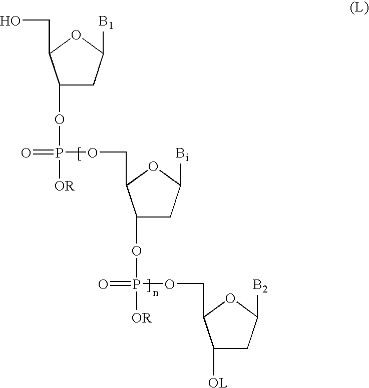
where B₁, B₂, Bᵢ can be H, adeninyl, cytosinyl, guaninyl, thyminyl, uracilyl, 2,6-diaminopurine-9-yl, hypoxanthine-9-yl, 5-methylcytosine-1-yl, 5-amino-4-carboxylimidazol-1-yl or 5-amino4-carbamoylimidazol-1-yl independently from each other, where in the case of B₁, B₂, Bᵢ having primary amino functions, these may have a permanent protecting group, resp. with thyminyl or uracilyl at the O₄-position these can have a permanent protecting group if necessary, where R can be an H, alkyl, cycloalkyl, aryl, aralkyl, cyanoalkyl, haloalkyl rest, and where L stands for NPPOC, FMOC and NPC, and n=0 or is an integer from 1 to 4.

12. Nucleotide derivatives with the general formula (E): 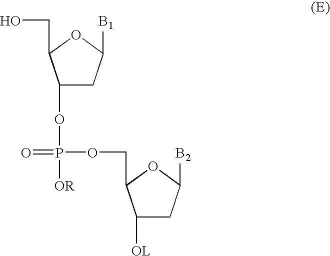
, where B₁ and B₂ can be H, adeninyl, cytosinyl, guaninyl, thyminyl, uracilyl, 2,6-diaminopurine-9-yl, hypoxanthine-9-yl, 5-methylcytosine-1-yl, 5-amino-4-caboxylimidazol-1-yl or 5-amino-4-carbamoylimidazol-1-yl independently from each other, where in the case of B₁, B₂ having primary amino functions these may have a permanent protecting group resp. with thyminyl or uracilyl a the O₄-position, these may have a permanent protecting group if necessary.
where R can be an H, alkyl, cycloalkyl, aryl, aralkyl, haloalkyl, cyanoalkyl rest,
and where L stands for NPPOC, FMOC and NPC.

13. Use of a nucleotide derivative according to claim 11 in a process according to claim 1 or 2.

14. Nucleotide derivative with the general formula (M) 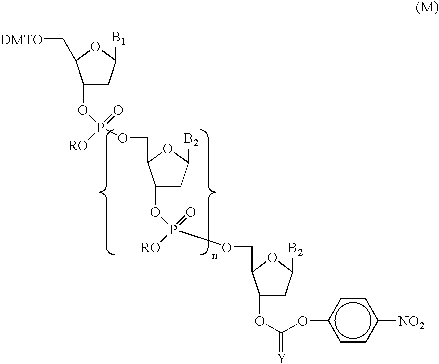
where B₁, B₂, Bᵢ can be adeninyl, cytosinyl, guaninyl, thyminyl, uracilyl, 2,6-diaminopurine-9-yl, hypoxanthine-9-yl, 5-methylcytosine-1-yl, 5-amino-4-carboxylimidazol-1-yl or 5-Amino-4-carbamoylimidazol-1-yl independently from each other, where in the case of B₁, B₂, Bᵢ having primary amino functions, these may have a permanent protecting group resp. with thyminyl or uracilyl at the O₄-position, these may have a permanent protecting group, if necessary,
where can be an H, an alkyl, cycloalkyl, aryl, aralkyl, haloalkyl, cyanoalkyl rest,
and Y═O or S and n=0 or an integer from 1 to 4.

15. Nucleotide derivative with the general formula (J) 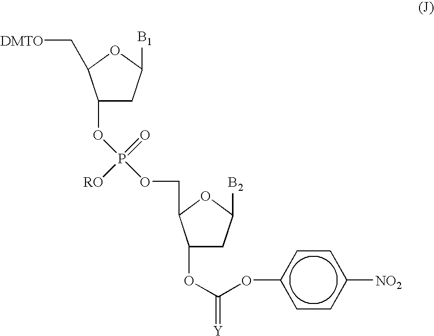
where B₁ and B₂ can be adeninyl, cytosinyl, guaninyl, thyminyl, uracilyl, 2,6-diaminopurine-9-yl, hypoxanthine-9-yl, 5-methylcytosine-1-yl, 5-amino-4-caboxylimidazol-1-yl or 5-amino-4-carbamoylimidazol-1-yl independently from each other, where in the case of B₁, B₂ having primary amino functions, these may have a permanent protecting group resp. with thyminyl or uracilyl at the O₄-position, these may have a permanent protecting group, if necessary,
where R can be H, an alkyl, cycloalkyl, aryl, aralkyl, haloalkyl, cyanoalkyl rest,
and Y═O or S.

16. Use of a nucleotide derivative according to claim 14 in a process according to claim 1 or 2.

17. Kit, which contains part of or all reagents and/or auxiliaries, for carrying out a process according to claim 1 or 2 in one unit, characterized in that the kit contains at least one or more selected nucleotide derivatives (E) and (J), which have a free 5′-hydroxy group and a protected 3′-hydroxy group and/or a suitable reagent for the introduction of the phosphate group.

18. Use of a process according to claim 1 or 2 and/or a kit according to claim 17 for the preparation of at least oneof oligonucleotides and nucleic acid chips.

19. Use of a process according to claim 1 or 2 and/or a kit according to claim 17 for the automated preparation of at least one of oligonucleotides and nucleic acid chips.

20. The process of claim 4 wherein the photolabile protecting group is at least one of NPPOC, MeNPOC, NVOC, PyMOC, NBOC, NPES and NPPS.

21. The process of claim 8 wherein the nucleic acid analog is at least one of PNA, LNA and chimeras thereof.

22. Process according to claim 10, wherein further mononucleotides are selected specifically or at random.

23. Use of a nucleotide derivative according to claim 12 in a process according to claim 1 or 2.

24. Use of a nucleotide derivative according to claim 15 in a process according to claim 1 or 2.

25. The Kit of claim 17 wherein the kit further comprises reagents or auxiliaries suitable for carrying out the process.

26. The Kit of claim 17 wherein the kit further comprises a work instruction suitable for carrying out the process.

27. Kit for carrying out a process according to claim 1 or 2 in one unit, characterized in that the kit contains at least one or more of the nucleotide derivatives (E), (J), (L) and (M).

28. The Kit of claim 27 wherein the kit further comprises reagents or auxiliaries suitable for carrying out the process.

29. The Kit of claim 27 wherein the kit further comprises a work instruction suitable for carrying out the process.
